(19) United States
(12) Patent Application Publication
(10) Pub. No.: US 2024/0197600 A1
(43) Pub. Date: Jun. 20, 2024

(54) Mouthwash Compositions and Methods

(71) Applicant: Colgate-Palmolive Company, New York, NY (US)

(72) Inventors: Nihal DOGU, Dayton, NJ (US); Betty WON, Princeton Junction, NJ (US); Paloma PIMENTA, Staten Island, NY (US); Lyndsay SCHAEFFER-KORBYLO, Flemington, NJ (US); Joseph Allan McKinnon STEELE, Plainfield, NJ (US); Lisa Marie MANUS, Lawrenceville, NJ (US); Lauren EVANS, Highland Park, NJ (US); Michael NORTH, Middlesex, NJ (US)

(21) Appl. No.: 18/531,033

(22) Filed: Dec. 6, 2023

Related U.S. Application Data
(60) Provisional application No. 63/430,610, filed on Dec. 6, 2022.

(51) Int. Cl. A61K 8/46; A61Q 11/00

(52) U.S. Cl. CPC A61K 8/466; A61Q 11/00; A61K 2800/52; A61K 2800/592; A61K 2800/92

(57) ABSTRACT

This application relates to novel anti-bacterial mouthwash compositions comprising hyaluronic acid, cetyl pyridinium chloride, and taurate surfactant, having unexpected stability, and their use in treating plaque, gingivitis, bad breath, implant infections, tooth decay, inflammation, and dental staining, as well as helping to moisturize and protect the teeth and gums.

### FIELD

This application relates to novel anti-bacterial mouthwash compositions comprising hyaluronic acid, cetyl pyridinium chloride, and taurate surfactant, having unexpected stability, and their use in treating plaque, gingivitis, bad breath, implant infections and tooth decay and helping to moisturize and protect the teeth and gums.

### BACKGROUND

Dry mouth or xerostomia is an acute or chronic condition primarily caused by the lack of saliva. It may be caused by an underlying disease, such as Sjögren's syndrome, dehydration, trauma to the salivary glands, consumption of alcohol, or a side effect to medications. It has been identified as a condition increasing in the general population. Roughly 15% to 20% of young adults complain of oral dryness, and 30-40% of people ages 60-80 complain of oral dryness. Patients suffering from xerostomia may also suffer from extensive dental decay, e.g., caries, including areas not usually prone to decay, such as the lower incisors and roots. One possible explanation is that pellicle, which is present in saliva, provides a protective barrier between acids and a tooth surface, and such a barrier is reduced in the absence of saliva.

Oral surgery, including tooth extractions and implants, may also disrupt the oral environment. Dental implants can become infected, causing inflammation in the soft tissues and bone loss around the implant. Indeed, a high percentage of implants fail within the first year due to bacterial infection.

The most widely used antibacterial agent in mouthwashes in the United States, cetyl pyridinium chloride (CPC) presents challenges for formulations. Because cetyl pyridinium is strongly cationic, it may form salts or complexes with anionic compounds in the formulation. For example, hyaluronic acid (HA) is a natural ingredient providing moisturizing, anti-inflammatory, and barrier protection benefits for oral soft tissue, making it a desirable ingredient for use in mouthwash, but it is an acid, and thus has a potential for interaction with CPC. Developing stable mouthwash formulations comprising HA and CPC has proved challenging. Anionic surfactants such as sodium lauryl sulfate (SLS) are widely used in dentifrice formulations. SLS has the benefit, for example, of being neutral with respect to product taste and in a mouthwash, it may help solubilize flavoring agents. However, there has been recent consumer interest in developing various oral care products that do not contain SLS. Many mouthwashes also contain ethanol, which is antibacterial and helps solubilize ingredients that are poorly soluble in water, but ethanol may aggravate dry mouth and is disfavored by many consumers.

There is a need for improved mouthwashes which kill undesirable bacteria that cause plaque, gingivitis, bad breath, implant infections and tooth decay and which also help moisturize and protect the teeth and gums.

### SUMMARY OF THE INVENTION

Various anti-bacterial mouthwash formulations tested with the addition of 0.10% HA to a commercial mouthwash backbone containing 0.075% cetylpyridinum chloride (CPC) and 0.28% zinc lactate(ZnLac) fail stability testing, e.g. freeze-thaw cycling, due to formation of cetylpyridinum hyaluronate precipitate.

While anionic surfactants could potentially form stable micelles with positively charged CPC, preventing interaction with HA, such surfactants might interfere with CPC delivery to the teeth. Various surfactants were screened for their ability to inhibit formation of the CPC-HA complex. Availability of CPC was tested by measuring CPC uptake and anti-bacterial activity in short interval kill tests (SIKTs).

Some surfactants are simply ineffective at inhibiting formation of the CPC-HA complex. PEG40-hydrogenated castor oil, which is used as a surfactant in several mouthwash formulations containing HA, inhibits formation of the CPC-HA complex, but also interferes with CPC uptake. Surprisingly, however, sodium methyl coco taurate is effective both to inhibit formation of the CPC-HA complex and to enhance CPC uptake.

Accordingly, the disclosure provides anti-bacterial mouthwash compositions comprising hyaluronic acid, cetyl pyridinium chloride, and taurate surfactant, and the use of such compositions to treat and inhibit plaque, gingivitis, bad breath, implant infections, and tooth decay and to help moisturize and protect the teeth and gums.

Further areas of applicability of the present invention will become apparent from the detailed description provided hereinafter. It should be understood that the detailed description and specific examples, while indicating the preferred embodiment of the invention, are intended for purposes of illustration only and are not intended to limit the scope of the invention.

### DETAILED DESCRIPTION

The following description of the preferred embodiment(s) is merely exemplary in nature and is in no way intended to limit the invention, its application, or uses.

As used throughout, ranges are used as shorthand for describing each and every value that is within the range. Any value within the range can be selected as the terminus of the range. In addition, all references cited herein are hereby incorporated by referenced in their entireties. In the event of a conflict in a definition in the present disclosure and that of a cited reference, the present disclosure controls.

Unless otherwise specified, all percentages and amounts expressed herein and elsewhere in the specification should be understood to refer to percentages by weight relative to the total composition. The amounts given are based on the active weight of the material.

As is usual in the art, the compositions described herein are sometimes described in terms of their ingredients, notwithstanding that the ingredients may disassociate, associate or react in the formulation. Ions, for example, are commonly provided to a formulation in the form of a salt, which may dissolve and disassociate in aqueous solution. It is understood that the invention encompasses both the mixture of described ingredients and the product thus obtained.

It is understood that all ingredients in the compositions described herein are safe and palatable in the relevant concentrations for oral administration as a mouthwash.

In a first aspect, the present disclosure provides an antibacterial mouthwash composition (Composition 1) comprising hyaluronic acid (HA), cetyl pyridinium chloride (CPC), an effective amount of a taurate surfactant represented by Formula (1):

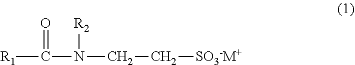

wherein R₁ is a saturated or unsaturated, straight or branched C₇₋₁₇ alkyl, R₂ is H or methyl, and M⁺ is H, sodium, or potassium (e.g., wherein the taurate surfactant is sodium methyl cocoyl taurate); and at least 70% water, by weight of the composition. For example, the disclosure provides embodiments of Composition 1 as follows:
1.1 Composition 1, wherein the taurate surfactant is sodium methyl cocoyl taurate.
1.2 Any foregoing composition wherein the taurate surfactant is sodium methyl cocoyl taurate in an amount of 0.3% to 0.8% by weight.
1.3 Any foregoing composition wherein the taurate surfactant is sodium methyl cocoyl taurate in an amount of 0.4% to 0.5% by weight.
1.4 Any foregoing composition wherein the amount of CPC is 0.05 to 0.1% by weight.
1.5 Any foregoing composition wherein the amount of CPC is about 0.075% by weight
1.6 Any foregoing composition wherein the HA is provided in the form of sodium hyaluronate.
1.7 Any foregoing composition wherein the amount of HA corresponds to 0.05 to 0.5% by weight of sodium hyaluronate.
1.8 Any foregoing composition wherein the amount of HA corresponds to 0.1% to 0.4% by weight of sodium hyaluronate.
1.9 Any foregoing composition wherein the amount of HA corresponds to 0.05 to 0.15% by weight of sodium hyaluronate.
1.10 Any foregoing composition wherein the amount of HA corresponds to about 0.1% by weight of sodium hyaluronate.
1.11 Any foregoing composition wherein the average molecular weight of the HA is at least 5 kDa.
1.12 Any foregoing composition wherein the average molecular weight of the HA is at least 100 kDa.
1.13 Any foregoing composition wherein the average molecular weight of the HA is at least 300 kDa.
1.14 Any foregoing composition wherein the average molecular weight of the HA is between 5 kD and 1 MDa.
1.15 Any foregoing composition wherein the average molecular weight of the HA is between 300 kD and 1 MDa.
1.16 Any foregoing composition wherein the HA is in the form of sodium hyaluronate average MW>100,000 Da, e.g., 300 kDa-1 MDa.
1.17 Any foregoing composition, further comprising a zinc ion source.
1.18 The foregoing composition wherein the zinc in source is an orally-acceptable soluble zinc salt, e.g. selected from zinc lactate, zinc citrate, and combinations thereof.
1.19 Any foregoing composition, further comprising a zinc salt wherein the zinc salt is zinc lactate.
1.20 Any foregoing composition, further comprising a zinc salt wherein the zinc salt is zinc lactate in an amount of 0.1% to 0.5%, e.g., 0.2% to 0.4% by weight.
1.21 Any foregoing composition, further comprising a zinc salt wherein the zinc salt is zinc lactate corresponding to zinc L-lactate dihydrate in an amount of about 0.28% by weight.
1.22 Any foregoing composition, further comprising a zinc salt wherein the zinc salt is zinc lactate in an amount of 0.2% to 0.4% by weight, and further comprising lactic acid in an amount of 0.4% to 0.6% by weight.
1.23 Any foregoing composition further comprising a fluoride ion source, e.g., sodium fluoride.
1.24 Any foregoing composition further comprising a fluoride ion source which is sodium fluoride in an amount of 0.04% to 0.06% by weight.
1.25 Any foregoing composition further comprising a nonionic surfactant.
1.26 Any foregoing composition further comprising a nonionic surfactant, wherein the nonionic surfactant is a poloxamer.
1.27 Any foregoing composition further comprising a nonionic surfactant, wherein the nonionic surfactant is poloxamer 407 in the amount of 0.3% to 0.5% by weight.
1.28 Any foregoing composition, further comprising a humectant, e.g selected from propylene glycol, glycerin, sorbitol, and combinations thereof.
1.29 Any foregoing composition, further comprising a humectant, wherein the humectant comprises propylene glycol, glycerin, and sorbitol in a total amount of 10% to 25% by weight.
1.30 Any foregoing composition, further comprising flavorings and sweeteners, e.g. selected from saccharin, sucralose, and combinations thereof.
1.31 Any foregoing composition, further comprising flavorings and sweeteners, wherein the sweetener comprises a combination of saccharin and sucralose.
1.32 Any foregoing composition, further comprising flavorings and sweeteners, wherein the sweetener comprises a combination of sodium saccharin in an amount of 0.01% to 0.03% by weight and sucralose in an amount of 0.001% to 0.003% by weight.
1.33 Any foregoing composition further comprising a preservative.
1.34 Any foregoing composition further comprising a preservative wherein the preservative comprises potassium sorbate in an amount of 0.04% to 0.06% by weight.
1.35 Any foregoing composition further comprising a preservative wherein the preservative comprises sodium benzoate in an amount of 0.01% to 0.15% by weight, e.g., about 0.05% by weight or about 0.1% by weight.
1.36 Any foregoing composition comprising food coloring, e.g., one or more orally acceptable water-soluble dyes, e.g., one or more FD&C water-soluble dyes.
1.37 Any foregoing composition wherein all of the ingredients are safe and palatable in the relevant concentrations for oral administration as a mouthwash.
1.38 Any foregoing composition wherein the pH is between pH 4 and pH 5.
1.39 Any foregoing composition wherein the delivery of CPC to the teeth is enhanced relative to a formulation having PEG-40 hydrogenated castor oil in place of a taurate surfactant, e.g., in place of sodium methyl cocoyl taurate.
1.40 Any foregoing composition wherein the antibacterial activity as measured in a short-interval kill test (SIKT) is superior to a formulation having PEG-40 hydrogenated castor oil in place of a taurate surfactant, e.g., in place of sodium methyl cocoyl taurate.
1.41 Any foregoing composition which does not form a cetylpyridinum hyaluronate precipitate in a freeze-thaw cycling test, e.g., freezing to −30° C. than thawing to 30° C.
1.42 Any foregoing composition which does not form a cetylpyridinum hyaluronate precipitate after four weeks of aging at 40° C.
1.43 Any foregoing composition which does not form a cetylpyridinum hyaluronate precipitate after four weeks of aging at −10° C.
1.44 Any foregoing composition which is free of ethanol.
1.45 Any foregoing composition which is free of PEG-40 hydrogenated castor oil.
1.46 Any foregoing composition which is free of betaine surfactants.
1.47 Any foregoing composition which is free of cocamidopropyl betaine.
1.48 Any foregoing composition which is free of sodium coco glutamate.
1.49 Any foregoing composition which is free of decyl glucoside.
1.50 Any foregoing composition which is free of sodium lauryl sulfate.
1.51 Any foregoing composition which is free of ionic surfactants other than taurate surfactants.
1.52 Any foregoing composition which is free of ionic surfactants other than is sodium methyl cocoyl taurate.
1.53 Any foregoing composition which is formed by combining ingredients comprising:
   0.05 to 0.15% sodium hyaluronate,
   0.05 to 0.1% cetyl pyridinium chloride,
   0.3% to 0.8% sodium methyl cocoyl taurate, and
   75% to 85% by weight water,
   and optionally further comprising one or more of zinc ion sources,
   humectants, non-ionic surfactants, fluoride ion sources,
   humectants, preservatives, sweeteners, flavorings, and/or dyes,
   wherein all percentages are by weight of the composition.
1.54 Any foregoing composition which is formed by combining ingredients comprising:
   0.05-0.15% sodium hyaluronate,
   0.07-0.08% cetyl pyridinium chloride,
   0.4-0.5% sodium methyl cocoyl taurate,
   0.2-0.4% zinc L-lactate dihydrate, and
   75-85% water,
   wherein all percentages are by weight of the composition.
1.55 Any foregoing composition which is formed by combining ingredients comprising:
   0.05-0.15% sodium hyaluronate,
   0.07-0.08% cetyl pyridinium chloride,
   0.4-0.5% sodium methyl cocoyl taurate,
   0.2-0.4% zinc L-lactate dihydrate,
   15-20% humectants comprising glycerin, propylene glycol, and sorbitol,
   0.3-0.5% poloxamer 407,
   0.04-0.06% L-lactic acid,
   0.04-0.06% sodium fluoride,
   0.02-0.03% sweetener comprising sucralose and sodium saccharin,
   0.05-0.2% flavoring comprising one or more cooling agents,
   0.01-0.15% preservatives, selected from potassium sorbate, sodium benzoate, and combinations thereof,
   0.00005 - 0.0005% water-soluble dye, and
   75-85% water,
   wherein all percentages are by weight of the composition.
1.56 Any foregoing composition for use as an antibacterial agent in the oral cavity.
1.57 Any foregoing composition for use in the treatment of xerostomia.
1.58 Any foregoing composition for use to treat and inhibit plaque.
1.59 Any foregoing composition for use to treat and inhibit gingivitis.
1.60 Any foregoing composition for use to treat and inhibit bad breath.
1.61 Any foregoing composition for use to treat and inhibit implant infections.
1.62 Any foregoing composition for use to treat and inhibit tooth decay.
1.63 Any foregoing composition for use to treat and inhibit caries.
1.64 Any foregoing composition for use to moisturize and protect the teeth and gums.
1.65 Any foregoing composition for use to reduce inflammation in the oral cavity.
1.66 Any foregoing composition for use to reduce staining of the teeth.
1.67 Any foregoing composition which does not form a visible precipitate following freeze thaw cycling (30° C. to −30° C. to 30° C.)
1.68 Any foregoing composition which is stable under the following storage conditions: 4 weeks at −10° C., 4 weeks at 40° C., and 4 weeks at 49° C., with respect to appearance, CPC levels, pH levels, and HA levels.
1.69 Any foregoing composition which exhibits antibacterial activity, e.g., provides at least 25% kill in a short-interval kill test, e.g., in a short-interval kill test substantially as described in Example 1.
1.70 Any foregoing composition which exhibits anti-inflammatory activity, e.g., reduces the release of IL-8 consequent to treatment with pro-inflammatory stimuli using IL-1alpha, e.g., in an assay substantially as described in Example 2.
1.71 Any foregoing composition which exhibits anti-staining activity, e.g., which reduces dental staining by coffee, tea, and wine, e.g. as demonstrated in an assay substantially as described in Example 3.
1.72 Any foregoing composition comprising zinc, wherein the HA enhances the uptake of zinc relative to a formulation that is the same except that the HA is replaced with an equivalent amount of water.

In another aspect, the present disclosure further provides a method of inhibiting formation of cetylpyridinium hyaluronate precipitate in a mouthwash comprising cetylpyridinium chloride and hyaluronic acid, comprising adding a taurate surfactant, e.g., to form a mouthwash composition according to any of Compositions 1, et. seq.

In another aspect, the present disclosure provides the use of a taurate surfactant in the manufacture of a mouthwash comprising cetylpyridinium chloride and hyaluronic acid to inhibit formation of cetylpyridinium hyaluronate precipitate, e.g., wherein the mouthwash is a mouthwash composition according to any of Compositions 1, et. seq.

In another aspect, the present disclosure provides a method of treatment or prophylaxis of one or more of xerostomia, plaque, gingivitis, bad breath, dental implant infections, tooth decay, and/or caries comprising administering to the oral cavity of a subject in need thereof a composition according to any of Compositions 1, et seq., for example, one or more times per day.

In another aspect, the present disclosure provides a method of reducing inflammation in the oral cavity comprising administering to the oral cavity of a subject in need thereof a composition according to any of Compositions 1, et seq., for example, one or more times per day.

In another aspect, the present disclosure provides a method of reducing dental staining comprising administering to the teeth of a subject in need thereof a composition according to any of Compositions 1, et seq., for example, one or more times per day.

In another aspect, the present disclosure provides a method of delivering zinc to the dental pellicle, comprising administering to the oral cavity of a subject in need thereof composition according to any of Compositions 1, et seq., wherein the composition comprises a zinc ion source; for example, administering one or more times per day.

The foregoing methods comprising administering a composition according to any of Compositions 1, et seq., to the oral cavity or teeth of a subject comprise applying any of the compositions as described herein to the teeth or oral cavity, e.g., by gargling or rinsing, or otherwise administering the compositions to the oral cavity of a subject in need thereof. The compositions can be administered regularly, such as, for example, one or more times per day (e.g., twice per day).

In various embodiments, administering the compositions of the present disclosure to teeth may provide one or more of the following specific benefits: (i) reduce or inhibit formation of dental caries, (ii) reduce, repair or inhibit pre-carious lesions of the enamel, e.g., as detected by quantitative light-induced fluorescence (QLF) or electrical caries measurement (ECM), (iii) reduce or inhibit demineralization and promote remineralization of the teeth, (iv) reduce hypersensitivity of the teeth, (v) reduce or inhibit gingivitis, (vi) promote healing of sores or cuts in the mouth, (vii) reduce levels of acid producing and/or malodor producing bacteria, (viii) treat, relieve or reduce dry mouth, (ix) clean the teeth and oral cavity, (x) whiten the teeth, (xi) reduce tartar build-up, (xii) reduce or prevent oral malodor, (xiii) reduce staining, (xiv) reduce inflammation, and/or (xv) promote systemic health, including cardiovascular health, e.g., by reducing potential for systemic infection via the oral tissues.

In another aspect, the present disclosure provides the use of a taurate surfactant in the manufacture of a mouthwash comprising cetylpyridinium chloride and hyaluronic acid, e.g., any of Compositions 1, et seq. for treating or preventing one or more of xerostomia, plaque, gingivitis, bad breath, dental implant infections, tooth decay, and/or caries.

As used herein, an “oral care composition” refers to a composition for which the intended use includes oral care, oral hygiene, and/or oral appearance, or for which the intended method of use comprises administration to the oral cavity, and refers to compositions that are palatable and safe for topical administration to the oral cavity, and for providing a benefit to the teeth and/or oral cavity. The term “oral care composition” thus specifically excludes compositions which are highly toxic, unpalatable, or otherwise unsuitable for administration to the oral cavity. In some embodiments, an oral care composition is not intentionally swallowed, but is rather retained in the oral cavity for a time sufficient to affect the intended utility. The oral care compositions as disclosed herein may be used in nonhuman mammals such as companion animals (e.g., dogs and cats), as well as by humans. In some embodiments, the oral care compositions as disclosed herein are used by humans. Oral care compositions include, for example, dentifrice and mouthwash. In some embodiments, the disclosure provides mouthwash formulations.

Weight percentages as used herein are by weight of the composition, unless otherwise indicated.

As used herein, “orally acceptable” refers to a material that is safe and palatable at the relevant concentrations for use in an oral care formulation, such as a mouthwash.

As used herein, “nonionic surfactant” generally refers to compounds produced by the condensation of alkylene oxide groups (hydrophilic in nature) with an organic hydrophobic compound which may be aliphatic or alkyl-aromatic in nature. Examples of suitable nonionic surfactants include poloxamers (sold under trade name PLURONIC®), polyoxyethylene, polyoxyethylene sorbitan esters (sold under trade name TWEENS®), Polyoxyl 40 hydrogenated castor oil, fatty alcohol ethoxylates, polyethylene oxide condensates of alkyl phenols, products derived from the condensation of ethylene oxide with the reaction product of propylene oxide and ethylene diamine, ethylene oxide condensates of aliphatic alcohols, alkyl polyglycosides (for example, fatty alcohol ethers of polyglycosides, such as fatty alcohol ethers of polyglucosides, e.g., decyl, lauryl, capryl, caprylyl, myristyl, stearyl and other ethers of glucose and polyglucoside polymers, including mixed ethers such as capryl/caprylyl (C₈₋₁₀) glucoside, coco (C₈₋₁₆) glucoside, and lauryl (C₁₂₋₁₆) glucoside), long chain tertiary amine oxides, long chain tertiary phosphine oxides, long chain dialkyl sulfoxides, and mixtures of such materials.

In some embodiments, the nonionic surfactant comprises amine oxides, fatty acid amides, ethoxylated fatty alcohols, block copolymers of polyethylene glycol and polypropylene glycol, glycerol alkyl esters, polyoxyethytene glycol octylphenol ethers, sorbitan alkyl esters, polyoxyethylene glycol sorbitan alkyl esters, and mixtures thereof. Examples of amine oxides include, but are not limited to, laurylamidopropyl dimethylamine oxide, myristylamidopropyl dimethylamine oxide, and mixtures thereof. Examples of fatty acid amides include, but are not limited to, cocomonoethanolamide, lauramide monoethanolamide, cocodiethanolamide, and mixtures thereof. In certain embodiments, the nonionic surfactant is a combination of an amine oxide and a fatty acid amide. In certain embodiments, the amine oxide is a mixture of laurylamidopropyl dimethylamine oxide and myristylamidopropyl dimethylamine oxide. In certain embodiments, the nonionic surfactant is a combination of lauryl/myristylamidopropyl dimethylamine oxide and cocomonoethanolamide. In certain embodiments, the nonionic surfactant is present in an amount of 0.01 to 5.0%, 0.1 to 2.0%, 0.1 to 0.6%, 0.2 to 0.4%, about 0.2%, or about 0.5%.

Mouthwashes frequently contain significant levels of ethanol, which is often needed to solubilize essential oils and to prevent bacterial contamination. High levels of ethanol may be undesirable, because in addition to the potential for abuse by ingestion, the ethanol may exacerbate conditions like xerostomia. Accordingly, in some embodiments, the oral care compositions of the invention are substantially free of ethanol, e.g., contain less than 1% ethanol.

Humectants can enhance the viscosity, mouthfeel, and sweetness of the product, and may also help preserve the product from degradation or microbial contamination. Suitable humectants include edible polyhydric alcohols such as glycerin, sorbitol, xylitol, propylene glycol as well as other polyols and mixtures of these humectants. Sorbitol may in some cases be provided as a hydrogenated starch hydrolysate in syrup form, which comprises primarily sorbitol (the product if the starch were completely hydrolyzed to glucose, then hydrogenated), but due to incomplete hydrolysis and/or presence of saccharides other than glucose, may also include other sugar alcohols such mannitol, maltitol, and longer chain hydrogenated saccharides, and these other sugar alcohols also function as humectants in this case. In some embodiments, humectants are present at levels of 5% to 25%, e.g., 15% to 20% by weight.

Flavorings for use in the present invention may include extracts or oils from flavorful plants such as peppermint, spearmint, cinnamon, wintergreen, and combinations thereof, cooling agents such as menthol, methyl salicylate, and commercially available products such as OptaCool® from Symrise. Sweeteners for use in the present invention (in addition to polyols, which also function as humectants) are preferably non-saccharide sweeteners, e.g., selected from saccharin, acesulfame, aspartame, neotame, stevia and sucralose.

Other ingredients which may optionally be included in compositions according to the present invention include green tea, ginger, sea salt, coconut oil, turmeric, white turmeric (white curcumin), grape seed oil, ginseng, monk fruit, vitamin E, basil, chamomile, pomegranate, aloe vera, and charcoal. Any of such ingredients may be present in an amount from 0.01% to 2% by weight of the composition, e.g., 0.01 to 1%, or 0.01 to 0.5%, or 0.01 to 0.1%.

### EXAMPLES

### Example 1—Formulation Screening

Mouthwash formulations comprising SMCT, CPC, and HA are prepared as follows:

TABLE 1

| | Description | | |
|---|---|---|---|
| | 0.10% HA, | | |
| | and Test | 0.10% HA, | 0.10% HA, |
| | surfactants | 0.45% SMCT | 0.70% SMCT |
| Ingredients | Wt % | Wt % | Wt % |
| DEMINERALIZED | 78-79 | 78-79 | 78-79 |
| WATER | | | |
| GLYCERIN - USP, EP | 7-8 | 7-8 | 7-8 |
| VEG | | | |
| PROPYLENE GLYCOL | 6.5-7.5 | 6.5-7.5 | 6.5-7.5 |
| USP, EP | | | |
| SORBITOL - NON- | 5-6 | 5-6 | 5-6 |
| CRYSTAL - 70% SOLN | | | |
| USP, EP | | | |
| SODIUM METHYL | — | 0.45 | 0.7 |
| COCOYL TAURATE* | | | |
| POLOXAMER 407 | 0.3-0.5 | 0.3-0.5 | 0.3-0.5 |
| USP-NF, EP | | | |
| Zinc L-lactate dihydrate | 0.28 | 0.28 | 0.28 |
| Cooling flavor | 0.1 | 0.1 | 0.1 |
| Sodium Hyaluronate | 0.1 | 0.1 | 0.1 |
| FCC grade | | | |
| CETYLPYRIDINIUM | 0.075 | 0.075 | 0.075 |
| CHLORIDE USP | | | |
| L-LACTIC ACID, 85% | 0.05-0.06 | 0.05-0.06 | 0.05-0.06 |
| SODIUM FLUORIDE - | 0.05 | 0.05 | 0.05 |
| USP, EP | | | |
| POTASSIUM | 0.05 | 0.05 | 0.05 |
| SORBATE - USP EP | | | |
| SODIUM SACCHARIN | 0.01-0.03 | .01-0.03 | .01-0.03 |
| USP, EP | | | |
| SUCRALOSE USP, EP | 0.001-0.003 | 0.001-0.003 | 0.001-0.003 |
| Coloring | 0.0001 | 0.0001 | 0.0001 |
| Test surfactant | 0.3-0.7 | — | — |
| Total Components | 100.000000 | 100.000000 | 100.000000 |

The ingredients and amounts in the formulations are the same except for the sodium cocyl methyl taurate and the test surfactants. The amount of water is adjusted wt/wt to account for the different amounts of surfactant. The test surfactants are as follows:
A. 0.3% PEG-40 hydrogenated castor oil
B. 0.45% sodium cocoyl glutamate
C. 0.7% sodium cocoyl glutamate
D. 0.45% decyl glucoside
E. 0.7% decyl glucoside
F. 0.45% cocamidopropyl betaine
G. 0.7% cocamidopropyl betaine

Formulations containing either PEG-40 hydrogenated castor oil or sodium methyl cocoyl taurate passed freeze thaw cycling (30° C. to −30° C. to 30° C.). A precipitate was seen immediately after defrosting the PEG 40 formula; however the precipitate re-solubilized within 24 hours. A CPC uptake test was conducted on HAP discs to test for availability of CPC. The two formulations containing sodium methyl cocoyl taurate delivered significantly higher CPC uptake than the formulation with PEG40 hydrogenated castor oil, and there was no CPC delivery at all with the other surfactants, due to the formation of cetylpyridinium hyaluronate precipitate precipitate.

TABLE 2

| | Surfactants Screened | | | | | |
|---|---|---|---|---|---|---|
| | | 0.45, | | | 0.45% | 0.7% |
| | 0.30% | 0.7% | 0.45, | 0.45, | Sodium | Sodium |
| | PEG40 | Sodium | 0.7% | 0.7% | Methyl | Methyl |
| | Hydrogenated | cocoyl | Decyl | CAP | Cocoyl | Cocoyl |
| | castor oil | glutamate | Glucoside | Betaine | Taurate | Taurate |
| Freeze- | Precipitate | Fail | Fail | Fail | Pass | Pass |
| Thaw | forms but re- | | | | | |
| Stability | solubilizes | | | | | |
| CPC | Poor Uptake | — | — | — | Good | Good |
| Uptake | (0.004 ppm) | | | | uptake | uptake |
| (HAP disc) | | | | | (0.027 ppm) | (0.020 ppm) |

For comparison, two commercial mouthwash formulations comprising CPC are tested as positive controls. One exhibited 0.014 ppm uptake of CPC on the HAP discs; the other exhibited 0.009 ppm uptake of CPC on the HAP discs. The formulations comprising hyaluronic acid, CPC and sodium methyl cocoyl taurate thus delivered CPC better than the identical formulations with different surfactants, and also better than commercial formulations tested.

The formulation with 0.45% sodium methyl cocoyl taurate was then tested for antibacterial activity against the formulation with 0.30% PEG40 hydrogenated castor oil in a short-interval kill test. The percent kill for the 0.45% sodium methyl cocoyl taurate formulation was 28.47% vs. 22.79% kill for the 0.30% PEG40 hydrogenated castor oil, showing that (as expected) enhanced CPC delivery correlates with enhanced antibacterial activity.

The above-described sodium methyl cocoyl taurate formulations are then further evaluated with different levels of additional preservative (0.05% and 0.1% by weight sodium benzoate) and shown to be stable in the freeze-thaw cycling assay and under different storage conditions (4 wk at −10° C., 4 wk at 40° C. and 4 wk at 49° C.), with respect to appearance, CPC levels, NaF levels, pH levels, and HA (as detected by ELISA).

TABLE 3A
0.10% HA/0.45% SMCT/0.05% sodium benzoate

| | Time Points | | | | |
|---|---|---|---|---|---|
| Stability | Initial | F/T | 4 wk | 4 wk | 4 wk |
| Testing | (30 C.) | (−30° C.) | (−10° C.) | (40° C.) | (49° C.) |
| Cosmetic | Pass | Pass | Pass | Pass | Pass |
| Appearance | | | | | |
| CPC | 0.077% | 0.075% | 0.075% | 0.075% | 0.075% |
| Sodium | 224 ppm | 227 ppm | pending | pending | pending |
| Fluoride | | | | | |
| pH (as is) | 4.7 | 4.6 | 4.6 | 4.6 | 4.6 |
| HA Assay* | Detected | Detected | Detected | Detected | Detected |

TABLE 3B
0.10% HA/0.7% SMCT/0.05% sodium benzoate

| | Time Points | | | | |
|---|---|---|---|---|---|
| Stability | Initial | F/T | 4 wk | 4 wk | 4 wk |
| Testing | (30° C.) | (−30° C.) | (−10° C.) | (40° C.) | (49° C.) |
| Cosmetic | Pass | Pass | Pass | Pass | Pass |
| Appearance | | | | | |
| CPC | 0.076% | 0.075% | 0.075% | 0.074% | 0.075% |
| Sodium | 223 ppm | 225 ppm | pending | pending | pending |
| Fluoride | | | | | |
| pH (as is) | 4.7 | 4.6 | 4.7 | 4.6 | 4.6 |
| HA Assay* | Detected | Detected | Detected | Detected | Detected |

TABLE 3C
0.10% HA/0.45% SMCT/0.1% sodium
benzoate (only freeze-thaw assay)

Time Points

| Stability Testing | Initial (30° C.) | F/T (−30° C.) |
|---|---|---|
| Cosmetic Appearance | Pass | Pass |
| CPC | 0.075% | 0.075% |
| Sodium Fluoride | 225 ppm | 225 ppm |
| pH (as is) | 4.9 | 4.6 |
| HA Assay* | Detected | Detected |

### Example 2: Anti-inflammatory effects as measured in IL-8 release assay

Hyaluronic acid is a muco-adhesive polymer naturally found in soft periodontal tissue. Literature and external clinical research shows that Hyaluronic acid (HA) can provide anti-inflammatory and healing benefits for oral soft tissue. Hyaluronic acid (550 kDa Biomost, Bloomage Bioactive) is incorporated into a CPC/Zn mouthwash backbone at 0.10%. The anti-inflammatory test assesses the potential modulation of interleukin due to anti-inflammatory compounds, following treatment of pro-inflammatory stimuli (IL-1alpha) screening for readout of marker Interleukin 8 (IL-8).

Mouthwash formulations comprising SMCT, zinc lactate, and CPC, with and without HA, are prepared as in Example 1. The specific formulations are identical except for the presence or absence of HA; the amount of water is adjusted make up 100%.

TABLE 2

| | Description | |
|---|---|---|
| | Test Formulation A | Test Formulation B |
| | 0% HA | 0.10% HA |
| | 0.45% SMCT | 0.45% SMCT |
| | 0.28% zinc lactate | 0.28% zinc lactate |
| Ingredients | Wt % | Wt % |
| DEMINERALIZED | 78-79 | 78-79 |
| WATER* | | |
| GLYCERIN - USP, EP | 7-8 | 7-8 |
| VEG | | |
| PROPYLENE GLYCOL | 6.5-7.5 | 6.5-7.5 |
| USP, EP | | |
| SORBITOL - NON- | 5-6 | 5-6 |
| CRYSTAL - 70% SOLN | | |
| USP, EP | | |
| SODIUM METHYL | 0.45 | 0.45 |
| COCOYL TAURATE* | | |
| POLOXAMER 407 | 0.3-0.5 | 0.3-0.5 |
| USP-NF, EP | | |
| Zinc L-lactate dihydrate | 0.28 | 0.28 |
| Cooling flavor | 0.1 | 0.1 |
| Sodium Hyaluronate | 0.1 | 0.1 |
| FCC grade | | |
| CETYLPYRIDINIUM | 0.075 | 0.075 |
| CHLORIDE USP | | |
| L-LACTIC ACID, 85% | 0.05-0.06 | 0.05-0.06 |
| SODIUM FLUORIDE - | 0.05 | 0.05 |
| USP, EP | | |
| POTASSIUM | 0.05 | 0.05 |
| SORBATE - USP EP | | |
| SODIUM SACCHARIN | .01-0.03 | .01-0.03 |
| USP, EP | | |
| SUCRALOSE USP, EP | 0.001-0.003 | 0.001-0.003 |
| Coloring | 0.0001 | 0.0001 |
| Total Components | 100.000000 | 100.000000 |

Human skin from abdominal plastic surgery is used (donor: female, born 1981). Skin samples are cut in pieces of approximately 8×3 mm thickness and cultured. Organ culture of ex-vivo human skin is used as a substrate to assess the modulatory activity of skin samples on Interleukin 8 levels in the presence of IL-1alpha. Read out of Interleukin 8 is measured using an ELISA test. On day 1, the skin samples are acclimated to the culture medium. On day 2, the test samples are applied before and during incubation with IL-1alpha. The skin samples are gently cleaned every time before applying the test samples, which are applied and covered with a 6 mm delivery membrane. On day 3, the organ cultures are withdrawn from the wells and analyzed for IL-8.

IL-8 is measured by absorbance 450-570 nm, and a standard curve is prepared to enable measurement of IL-8 in pg/ml. The concentration of IL-8 is measured in the presence of the two test formulations, and expressed as absolute concentration/weight of the tissue sample (abs/wt). In the presence of Test Formulation A, without HA, the average IL-8 release is 41.2 abs/wt. In the presence of Test Formulation B, with HA, the average IL-8 release is 21.4 abs/wt. Statistical analysis using a Tukey ANOVA test confirms the significance of this difference:

### ANOVA:

| Samples | N | Mean | StDev | 95% CI |
|---|---|---|---|---|
| 0.10% HA MW | 6 | 21.361 | 0.676 | (19.920, 22.802) |
| Placebo | 6 | 41.194 | 2.135 | (39.753, 42.635) |

### Grouping Information Using the Tukey Method and 95% Confidence

| Samples | N | Mean | Grouping |
|---|---|---|---|
| Placebo | 6 | 41.194 | A |
| 0.10% HA MW | 6 | 21.361 | B |

Means that do not share a letter are significantly different.

These results demonstrate that the addition of 0.10% Hyaluronic acid into the CPC/Zn mouthwash formula helps significantly to reduce the release of IL-8 consequent to treatment with pro-inflammatory stimuli using IL-1alpha. The test formula containing 0.10% hyaluronic acid helps reduce the modulation of IL-8 by 48% compared to the placebo mouthwash, 21.4 vs. 41.2 respectively. This highlights the benefit of hyaluronic acid to help reduce inflammation on soft tissue after exposure to inflammatory stimuli.

### Example 3—Stain Prevention Using Varying Molecular Weights and Amounts of HA

The impact of hyaluronic acid on whitening and stain prevention is assessed using different molecular weights of HA in mouthwashes comprising SMCT, CPC, and HA.

TABLE 3

| | Description |
|---|---|
| Ingredients | Wt % |
| DEMINERALIZED | 78-79 |
| WATER | |
| GLYCERIN - USP, EP | 7-8 |
| VEG | |
| PROPYLENE GLYCOL | 6.5-7.5 |
| USP, EP | |
| SORBITOL - NON- | 5-6 |
| CRYSTAL - 70% SOLN | |
| USP, EP | |
| SODIUM METHYL | 0.45 |
| COCOYL TAURATE | |
| POLOXAMER 407 | 0.3-0.5 |
| USP-NF, EP | |
| Cooling flavor | 0.1 |
| Sodium Hyaluronate | 0.1 |
| FCC grade | |
| CETYLPYRIDINIUM | 0.075 |
| CHLORIDE USP | |
| L-LACTIC ACID, 85% | 0.05-0.06 |
| SODIUM FLUORIDE - | 0.05 |
| USP, EP | |
| POTASSIUM | 0.05 |
| SORBATE - USP EP | |
| SODIUM SACCHARIN | 0.01-0.03 |
| USP, EP | |
| SUCRALOSE USP, EP | 0.001-0.003 |
| Coloring | 0.0001 |
| Total Components | 100.000000 |

Three variants of the above formulation are tested:
a) Low molecular weight HA (0.1% Sodium Hyaluronate, <5 kDa)
b) Medium molecular weight HA (Sodium Hyaluronate, 300 kDa
(c) High molecular weight HA (Sodium Hyaluronate, 1 MDa)

Baseline L, A, B, WIO measurements of bovine teeth are measured using a spectroshade micro II (Medical High Technologies) and split into treatment groups consisting of 6 teeth per group. After the baseline measurements each cell is placed into a separate test product (detailed below) for 60 seconds, afterwards the cells are exposed to a stain broth consisting of coffee, tea, and wine for 5 minutes. Following this, artificial saliva is placed into the cells for an additional five minutes. This product/stain/artificial saliva cycle is repeated 14 times; the product/stain/saliva is refreshed every cycle. Measurements are taken every two cycles. The teeth are patted dry prior to measuring the L, A, B. W values. Statistical significance is determined asing a Tukey ANOVA test. The following table depicts ΔW values for stain prevention efficacy of hyaluronic acid containing mouthwashes on bovine teeth. Letters (A,B) indicate values statistically different from each other. Lower numbers are better, indicating less staining.

| | HA | | | |
|---|---|---|---|---|
| | Molecular | Average delta | Standard | 95% |
| Sample | Weight | W | deviation | confidence |
| Control (water) | N/A | 17.99 (A) | 5.82 | 13.33, 22.65 |
| +0.1% Low | <5 kDa | 10.60 (A, B) | 3.19 | 8.05, 13.15 |
| Molecular Weight | | | | |
| HA | | | | |
| +0.1% Medium | 300 kDa | 7.86 (B) | 3.25 | 5.26, 10.46 |
| Molecular Weight | | | | |
| HA | | | | |
| +0.1% High | 1 MDa | 6.24 (B) | 0.73 | 5.60, 6.88 |
| Molecular Weight | | | | |
| HA | | | | |

There is a directional benefit of hyaluronic acid molecular weight on stain prevention. Medium and high molecular weight HA provide an anti-stain benefit at 14 cycles, relative to water alone. This may be attributed to the improved film-forming capabilities of higher molecular weight hyaluronate producing a stain shield of increased efficacy.

A further staining experiment is carried out, with the medium molecular weight HA at 0.05% HA, 0.1% HA, and 0.4% HA, using the formulation as described above except that the amount of water is adjusted to compensate for the amount of HA. The results are as follows:

| | Average delta | Standard | 95% |
|---|---|---|---|
| Sample | W | deviation | confidence |
| Control (water) | 18.7 (A) | 4.6 | 3.7 |
| 0.05% HA | 13.0 (B) | 2.5 | 2.0 |
| 0.1% HA | 8.7 (BC) | 1.8 | 1.4 |
| 0.4% HA | 6.9 (C) | 1.2 | 1.0 |

### Example 4: Effect of HA on Zinc Deposition

The addition of hyaluronic acid to oral care formulations containing zinc correlates with increased deposition of zinc on pellicle-coated oral surfaces in vitro, A pellicle coated Vitroskin model is used, where the Vitroskin discs are pre-incubated with artificial saliva to form a pellicle mimicking the dental pellicle, showing enhanced zinc deposition using 0.1% hyaluronic acid in a CPC-Zn mouthwash formulation.

In a mouthwash comprising zinc and CPC, with and without 0.1% HA, the HA actually reduces the level of soluble zinc slightly, but significantly increased zinc deposition on pellicle-coated Vitroskin in vitro models of oral mucosa:

| Formula | Soluble Zinc (ppm) | | Vitroskin Uptake (μg/cm²) | |
|---|---|---|---|---|
| Zn + CPC MW | 1023.5 ± 8.4 | A | 14.16 ± 0.21 | A |
| Zn + CPC + 0.1% HA | 985.9 ± 2.8 | B | 16.11 ± 0.95 | B |
| MW | | | | |

Formula that do not share a letter are statistically different (Tukey, 95% CI)

While the disclosure has been described with respect to specific examples including presently preferred modes of carrying out the disclosure, those skilled in the art will appreciate that there are numerous variations and permutations of the above-described systems and techniques. It is to be understood that other embodiments may be utilized and structural and functional modifications may be made without departing from the scope of the present disclosure.

1. An antibacterial mouthwash composition comprising hyaluronic acid (HA), cetyl pyridinium chloride (CPC), an effective amount of a taurate surfactant represented by Formula (1): 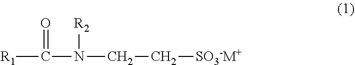
wherein R₁ is a saturated or unsaturated, straight or branched C₇₋₁₇ alkyl, R₂ is H or methyl, and M⁺ is H, sodium, or potassium; and at least 70% water, by weight of the composition.

2. The composition of claim 1, wherein the taurate surfactant is sodium methyl cocoyl taurate.

3. The composition of claim 2, wherein the taurate surfactant is sodium methyl cocoyl taurate in an amount of 0.3% to 0.8% by weight.

4. The composition of claim 1, wherein the amount of CPC is 0.05 to 0.1% by weight.

5. The composition of claim 1, wherein the amount of HA corresponds to 0.05 to 0.5% by weight of sodium hyaluronate.

6. The composition of claim 1, wherein the average molecular weight of the HA is between 300 kD and 1 MDa.

7. The composition of claim 1, further comprising a zinc salt.

8. The composition of claim 7, wherein the zinc salt is zinc lactate in an amount of 0.1% to 0.5%, by weight.

9. The composition of claim 1, further comprising a fluoride ion source.

10. The composition of claim 1, further comprising a nonionic surfactant.

11. The composition of claim 10, wherein the nonionic surfactant is poloxamer 407 in the amount of 0.3% to 0.5% by weight.

12. The composition of claim 1, further comprising a humectant.

13. The composition of claim 1, wherein the pH is between pH 4 and pH 5.

14. The composition of claim 1, wherein the delivery of CPC to the teeth is enhanced relative to a formulation having PEG-40 hydrogenated castor oil in place of a taurate surfactant, and/or wherein the antibacterial activity as measured in a short-interval kill test (SIKT) is superior to a formulation having PEG-40 hydrogenated castor oil in place of a taurate surfactant.

15. The composition of claim 1, which does not form a cetylpyridinum hyaluronate precipitate in a freeze-thaw cycling test, or after four weeks of aging at 40° C., or after four weeks of aging at −10° C.

16. The composition of claim 1 which is free of ethanol, PEG-40 hydrogenated castor oil, betaine surfactants, sodium lauryl sulfate, and/or any ionic surfactants other than sodium methyl cocoyl taurate.

17. The composition of claim 1 which is formed by combining ingredients comprising:
a. 0.05 to 0.15% sodium hyaluronate,
b. 0.05 to 0.1% cetyl pyridinium chloride,
c. 0.3% to 0.8% sodium methyl cocoyl taurate, and
d. 75% to 85% water,
and optionally further comprising one or more of zinc ion sources, humectants, non-ionic surfactants, fluoride ion sources, humectants, preservatives, sweeteners, flavorings, and/or dyes,
wherein all percentages are by weight of the composition.

18. The composition of claim 1 which is formed by combining ingredients comprising:
a. 0.05-0.15% sodium hyaluronate,
b. 0.07-0.08% cetyl pyridinium chloride,
c. 0.4-0.5% sodium methyl cocoyl taurate,
d. 0.2-0.4% zinc L-lactate dihydrate, and
e. 75-85% water,
wherein all percentages are by weight of the composition.

19. The composition of claim 1 which is formed by combining ingredients comprising:
a. 0.05-0.15% sodium hyaluronate,
b. 0.07-0.08% cetyl pyridinium chloride,
c. 0.4-0.5% sodium methyl cocoyl taurate,
d. 0.2-0.4% zinc L-lactate dihydrate,
e. 15-20% humectants comprising glycerin, propylene glycol, and sorbitol,
f. 0.3-0.5% poloxamer 407,
g. 0.04-0.06% L-lactic acid,
h. 0.04-0.06% sodium fluoride,
i. 0.02-0.03% sweetener comprising sucralose and sodium saccharin,
j. 0.05-0.2% flavoring comprising one or more cooling agents,
k. 0.01-0.15% preservatives, selected from potassium sorbate, sodium benzoate, and combinations thereof,
l. 0.00005 -0.0005% water-soluble dye, and
m. 75-85% water,
wherein all percentages are by weight of the composition.

20. (canceled)

21. A method of treatment or prophylaxis of one or more of xerostomia, plaque, gingivitis, bad breath, dental implant infections, tooth decay, and/or caries; or of reducing inflammation in the oral cavity; or of reducing dental staining, comprising administering to the oral cavity of a subject in need thereof a composition according to claim 1.

22. (canceled)

23. (canceled)

24. (canceled)
